(19) United States
(12) Patent Application Publication
(10) Pub. No.: US 2004/0131496 A1
(43) Pub. Date: Jul. 8, 2004

(54) Method and device at least for the sterilization of containers and/or the closing elements thereof

(76) Inventors: Bernd Goetzelmann, Rot Am See (DE); Johannes Rauschnabel, Stuttgart (DE); Hartmut Haegele, Schwieberdingen (DE); Bernd Wilke, Leutenbach (DE); Kurt Burger, Friolzheim (DE)

Correspondence Address: Ronald E Greigg, Greigg & Greigg, Suite One, 1423 Powhatan Street, Alexandria, VA, 22314, US

(21) Appl. No.: 10/478,147

(22) PCT Filed: Dec. 18, 2002
(86) PCT No.: PCT/DE02/04628

(30) Foreign Application Priority Data
Mar. 19, 2002 (DE) 102 11 976.7

(51) Int. Cl. B01J 19/08; B01J 19/12

(52) U.S. Cl. 422/22; 422/121; 422/186.04

(57) ABSTRACT

A method and an apparatus for sterilization, depyrogenization and/or annealing of containers and their closing elements (12) is proposed, in which in at least one method step, in a low-pressure chamber or vacuum chamber (4), a plasma treatment is performed jointly or separately for the containers and the closing elements (12) before the containers are filled, by means of excitation of an electromagnetic oscillation. The regions of the closing element or closing elements (12) and containers that are to be sterilized, depyrogenized and/or annealed are made to approach the oscillation-generating apparatus (15; 48) by means of a suitable conveyor device for one or more predetermined periods of time.

### PRIOR ART

The invention relates to a method at least for sterilizing containers and/or their closing elements, and an apparatus for performing the method, with a plasma treatment, based on the generic characteristics of the main and coordinate claims.

To eliminate harmful microorganisms or germs in medical containers or containers in the food industry, such as ampules, snap-top bottles, septum jars or so-called vials, or other so-called parenteral packs, it is well known to employ physical or chemical methods.

In the field of medical technology, a method is known, for instance from U.S. Pat. No. 5,961,921, in which medical instruments are sterilized in a vacuum by means of H₂O₂ vapor, and after that the peroxide residues are removed with a high-frequency plasma. A similar method is known in the field of beverage bottling, in which before being filled, so-called PET bottles are sterilized in a vacuum with H₂O₂, and the removal of the peroxide residues is accomplished by lessening the vacuum.

From U.S. Pat. No. 6,230,472, the sterilization of stoppers as closing elements for a container are also known, specifically in the context of a process in which the container, the filler needle, the closure, and a vacuum chamber are simultaneously sterilized using low-pressure and low-temperature plasma.

Furthermore, from International Patent Disclosure WO 96/13337, an apparatus for plasma coating of closing elements for vials is known, which is performed at low pressure inside a drum. Because of the drum geometry, the bulk material principle, and the discontinuous or batch process, this method is especially unsuited for modification in the direction of an aforementioned sterilization and of depyrogenization which is often necessary, and is moreover limited solely to coating the closure stoppers.

A particular disadvantage of these known methods is the complexity of the sterilization process, especially given the geometry of the elements to be sterilized, which have hollow shapes, undercuts, retainers, and so forth. Another disadvantage of the apparatuses known for the purpose is the sequential procedure for sterilizing, filling and closing the containers, which must be done here in the same chamber in all the work steps, yet because of the interplay of pressure, plasma excitation and filling with movable filler needles, these works have to be performed successively.

To assure both economical sterilization and a pyrogen depletion process in closable containers requires both short processing times and economical apparatuses. Furthermore, at least in the pharmaceutical field, these processes must be completely validatable by a predetermined standard and must to the fullest possible extent have what is known as in-situ process control.

In summary, the present practice, for instance for vials and injection bottles in medicine, contemplates above all that the sterilization and the annealing of the container and the closing element be done separately. The containers are for instance sterilized and depyrogenized using heat, while the closing elements, such as stoppers or septums, are as a rule acted upon with saturated steam at approximately 121° C. in an autoclave for sterilization. A significant pyrogen depletion cannot be accomplished in the autoclave. Even aqueous cleaning steps beforehand or afterward still fail to achieve adequate depletion. Chemical depyrogenization, for instance with strong acids or alkalis, undesirably alter the elastomers of the closing elements and are therefore hardly ever used.

The aforementioned autoclaving of the elastomer closing elements in the bulk material outside the insulator is also disadvantageous since as a rule the then-required delivery to the closing machine necessarily includes a separating station, where the production of harmful particles cannot be entirely precluded.

A method for sterilizing containers of the type recited above and an apparatus for performing the method with a plasma treatment of the containers in a vacuum chamber is also described in German Patent Disclosure DE 101 38 938, which had not yet been published by the priority date claimed for the present application.

Advantages of the Invention

With the invention, a method and an apparatus for sterilization and/or depyrogenization of closing elements for containers of the type defined at the outset, and in particular also in conjunction with the sterilization and/or depyrogenization of the containers themselves are advantageously further embodied by the characteristics of coordinate claims 1-3. This is accomplished using one or more plasmas, and in particular, low-pressure plasmas, plasmas near atmospheric pressure, or atmospheric plasmas can advantageously be employed here.

Thus in using the method of the invention, it is possible to design an apparatus that parallel to the process of sterilization, pyrogen depletion and optionally annealing for the containers also includes such a process for the closing elements as well, especially for closing elements made of elastomer materials, and thus overall makes completely validatable processes possible. In an application in the medical field or food industry, because of the permit procedures them required, and particularly also for flexibility in setting up a bottling line for special customer needs, the goal should be a modular concept of sterilizing the container and closing element for filling and closing purposes.

In particular, for the methods referred at the outset, it must be assured that all the regions and services of the closing elements are sterile when they are transferred into the bottling machine. There must be no possibility of cross-contamination of containers, closing elements, or handling elements. Any production of particles, for instance by impact or abrasion, must be avoided to the maximum possible extent, and according to the invention, a clear sterile boundary is attained in a packing line for the aforementioned containers, behind which everything either is sterile or can be kept sterile, and recontamination can be extensively precluded.

In claims 1-4, method characteristics are therefore disclosed with which in an especially advantageous way, a parallel treatment of the closing elements, especially elastomer stoppers, for container sterilization and pyrogen depletion is possible by exciting a plasma in a vacuum chamber, while adhering to the aforementioned peripheral conditions.

The closing elements are guided individually and sterilized on all sides along their course through the sterilization chamber, and pyrogens adhering to the closing element are depleted by decades, which is not possible with the usual known methods. Because of this extensively individualized handling, particle production is at least minimized, if not precluded. In sterilization in a vacuum chamber at pressures below 1 mbar, entrainment of particles into the filling machine can be nearly precluded. The individualized guidance of the closing elements also makes it possible to provide process control and process documentation for each element individually.

A parallel disposition of the closing element for container for container sterilization that is also advantageous for many applications, in a special vacuum chamber or adjacent special vacuum chambers, makes a smaller overall system size and a more-economical apparatus possible, compared to concepts in which the plasma sterilization, filling and closure stations are gone through sequentially in one and the same chamber.

After the inward transfer of the closing elements into the vacuum in the chamber, the elements must be sterilized and depyrogenized, and optionally also annealed or coated, on all sides. Because in the apparatus of the invention not only the closing elements but also the retention and conveyor devices are constantly exposed to a plasma, the sterility of these devices is assured as well, and cross-contamination before the outward transfer is avoided.

In the embodiments proposed in claims 5-9, retainers with an alternating contact region for the closing element surface via dies or needles are proposed as conveyor devices. However, a retainer can also be used that even in the contact region itself remains constantly sterile and also keeps the contact face of the closing element constantly sterile, for instance by means of a high temperature.

Since in this case the surfaces of the stoppers to be treated, for efficient sterilization, depyrogenization and/or annealing, can be brought in a simple way, by individualization and separate treatment, into the optimal position relative to the plasma source, it is possible to provide embodiments for a retainer in which, after a first step of sterilization of a region of the closing element, the closing element can then be grasped in a further step at precisely this now-sterile region, specifically by a sterile retainer, so that then the previous retainer and the region that is untreated until then can both be sterilized. However, this requires that no recontamination of the already sterile stopper region by the previous retainer be possible.

With conveyor devices proposed in the apparatus claims 10-18 as further advantageous embodiments, it is proposed that the closing element or elements are not retained fixedly but instead are guided in a defined, sterile way in a relatively loose retainer via a jigging table, rollers, or similar conveyor devices; as the closing elements pass through the plasma zone by sliding, rolling or hopping, they automatically arrive at an optimal position relative to the plasma.

According to dependent claims 19 and 20, various advantageous dispositions of the plasma source can be provided in the interior of the chamber; the conveyor device can itself be connected to an electrical terminal, such as the ground of the plasma source.

According to claim 21, it is also advantageously possible for the at least one plasma source, such as a so-called gigatron as a coaxial antenna, to be mounted in the interior of the chamber along the longitudinal axis of a rotating, slightly inclined quartz tube.

However, according to claim 22, the plasma source can also be mounted on the outside of the chamber and can be separated from the interior of the chamber by an arrangement by which the electromagnetic oscillations can be fed into the chamber and a pressure separation of the vacuum of the chamber from the pressure, deviating from it, outside the chamber can be accomplished.

### DRAWING

Exemplary embodiments of apparatuses for performing the method of the invention, particularly for sterilizing closing elements for containers, will now be described in conjunction with the drawing. Shown are:

FIG. 1, a block circuit diagram of a method sequence for the filling of containers, including the sterilization of closing elements for the containers and of the containers themselves;

FIG. 2, a first exemplary embodiment of a retainer with a vertically movable die for an individual closing element in the vacuum chamber for a plasma treatment;

FIG. 3, a second exemplary embodiment of a retainer with vertically movable needles for an individual closing element in the vacuum chamber;

FIG. 4, a third exemplary embodiment of a retainer with vertically movable needles that in a modification of FIG. 3 are inclined in alternation toward one another in groups;

FIG. 5, a fourth exemplary embodiment of a conveyor device as a loose retainer or guide for the closing elements, with a plate conveyor or jigger conveyor in the vacuum chamber;

FIGS. 6 and 7, a fifth exemplary embodiment of a conveyor device, comprising rollers, as a loose retainer or guide for the closing elements in the vacuum chamber, in views from different angles; and

FIGS. 8 and 9, a sixth exemplary embodiment of a conveyor device, comprising an inclined, rotating quartz tube with a plasma source inside it, in various views.

### DESCRIPTION OF THE EXEMPLARY EMBODIMENTS

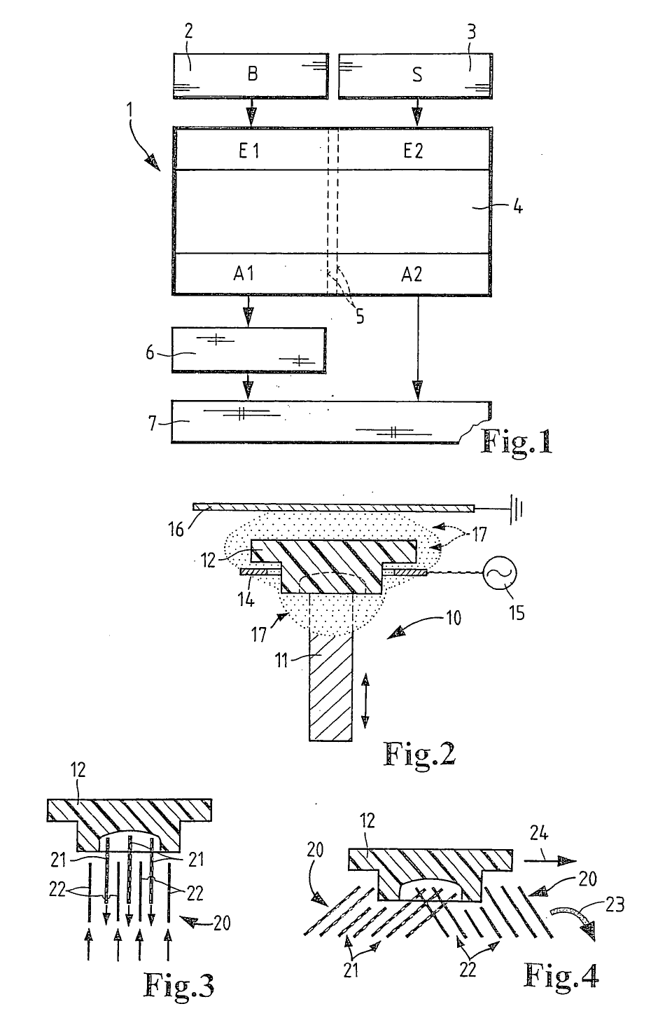

In FIG. 1, a block circuit diagram 1 is shown for a method for sterilizing and/or depyrogenizing of containers, not shown in detail here, in the medical or food industry, such as ampules, bottles or jars, and their closing elements, in particular stoppers made of an elastomer.

First, as shown in blocks 2 and 3 of FIG. 1, the containers B and stoppers S are separately precleaned, for instance washed, and then transferred inward into a low-pressure chamber or vacuum chamber E1 and E2, respectively, which is shown here as a single chamber, but as indicated by the dashed lines 5, the vacuum chamber 4 can also be constructed separately for the two process regions. Inside the chamber 4, for the actual sterilization and/or depyrogenization, a plasma treatment is then performed.

A plasma treatment of this kind can be effected by generating an electromagnetic oscillation in the range of a few Hertz up to the microwave range in the low-pressure chamber or vacuum chamber 4, or by means suitably performed inputting of waves into the chamber 4. Such plasma treatments are known per se from the prior art cited in the background section above for purposes of sterilization and/or annealing.

Following the plasma treatment, after the outward transfer at A1 and A2 of the containers and of the stopper as a closing element, respectively, from the chamber 4, filling of the container can be done in a filling machine in block 6. The filled container and the stopper for closing the container are then delivered to a closing station 7, in which the container can then be closed in its final form. The goal of the method is that especially during the method steps in the chamber 4, not only the containers and closing elements but also the retaining and conveyor devices are constantly exposed to a plasma, thus also assuring their sterility and preventing cross-contamination before the outward transfer. Suitable exemplary embodiments of such an apparatus will be described below.

In FIG. 2, the first exemplary embodiment of a conveyor device disposed in a vacuum chamber can be seen; it has a relatively solid retainer 10 with a vertically movable die 11 for receiving one elastomer stopper 12 at a time, the stopper being intended as a closing element for a container not shown here. The stopper 12 is placed with its bowl-shaped inside on an electrically conductive ring 14 in such a way that the downward-pointing inside fits into the ring 14. The ring 14 is subjected to a voltage of an oscillation generator 15, and the die 11 acts as a counterelectrode, being connected to ground, as is the surface 16 placed above the ring.

The output signal of the oscillation generator 15 is modulated in a manner known per se in terms of sign and amplitude to generate a plasma 17; in this exemplary embodiment, the modulation can range from a few Hertz to several hundred Megahertz, but preferably is between 50 kHz and 27 MHz. Oscillations in the radio frequency range of 13.56 MHz and 27 MHz are especially suitable here.

In a first step, after the stopper 12 has been deposited on the ring 14, all the sides of the stopper 12, except for the contact face between the ring 14 and the stopper 12, are subjected to the plasma 17, represented here by dots. In a second step, the grounded die 11 is moved vertically upward below the stopper 12, until it has lifted the stopper 12 several millimeters out of the ring 14. This then makes the sterilization, depyrogenizing or optionally even annealing of the previously untreated contact faces on the ring 14 and the stopper 12 possible.

This stopper 12, now sterilized on all sides, can be brought to the opening for outward transfer out of the chamber. The apparatus 10 of FIG. 2 can furthermore be improved by providing that the ring 14 has been coated with a thin, insulating polymer film, so that a replicably capacitive input of energy can be assured, while precluding electric arcs that often occur on bare metal faces of different polarity.

In an exemplary embodiment of FIG. 3, the stopper 12 is placed, in a modification of the retainer 10 of FIG. 2, on a needle cushion 20, in which a number of needles 21 as a group allow adequate stability of the retainer of the stopper 12, while another group 22 of other needles can perform the same task in a different method step.

The plasma is generated here in a comparable way to how it is done with the retainer 10 of FIG. 2 and subjects not only the needles 21, except for their area of contact with the stopper 12, but also the stopper 12 itself, again except for its area of contact with the needles 21, to the plasma. In order to treat these contact faces as well, the needles 22 of the further group are placed in a further step, by mutual vertical motion of the needles 21 and 22, below the stopper 12 in such a way that they then by themselves take on the task of retaining the stopper 12. The needles 21 that perform the retention before are then moved into a position in which they can be subjected to plasma, just as can the regions of the stopper 12 that had contact with retaining needles 21 of the first group and were accordingly shielded from the plasma.

The motion of the needles 21 and 22 can, as noted, be effected either by means of a linear motion in accordance with FIG. 3 and/or a motion of varying inclination in accordance with FIG. 4. With this type of motion (arrow 23) of the groups 21 and 22 of the needle cushion 20, a contrary and alternating inclination, optionally along with a linear motion, can be effected in such a way that the stopper 12 simultaneously executes a motion in the horizontal direction (arrow 24), and thus the apparatus functions as a so-called comb conveyor.

To assure subjection to plasma as much as possible on all sides of the stopper 12, electrical contacting of the needles of FIGS. 3 and 4 can be performed with the oscillation generator 15 (see FIG. 2). To avoid electrical contacts between a plurality of stoppers 12 in the processing line, these stoppers should traverse the sterilization, depyrogenization and annealing course individually or at a predetermined spacing, or in other words not in bulk.

In the above-described retainer of FIGS. 2-4, parallellization in the processing line can be accomplished by providing that instead of the ring 14 of FIG. 2, a perforated baffle and a die cushion, instead of a single die 11 along with a needle cushion, can be provided in the exemplary embodiments of FIGS. 3 and 4, to enable a high throughput along with secure electrical contacting.

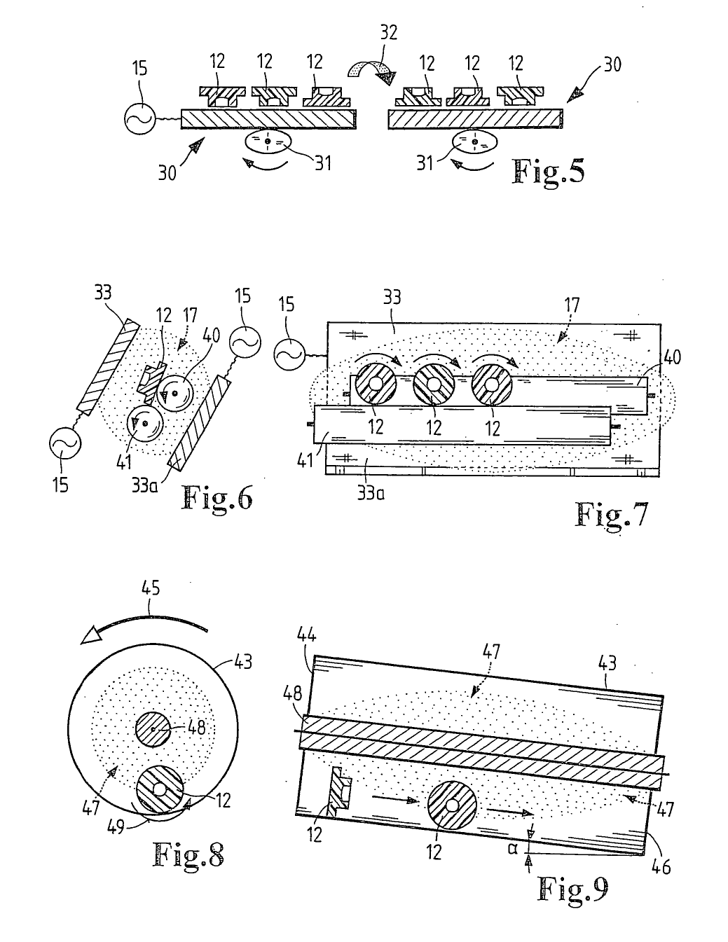

Instead of the comb conveyor principle of FIG. 4, a flat version of a retainer in FIG. 5 can be used. In this case the stoppers 12, after the inward transfer, are fed onto a surface 30, which may be embodied as a jigger table or plate conveyor, for instance with an eccentric motion device 31. The subjection to the plasma is done here from above, or if the surface 30 is embodied as a net or grating, it can also be done from below, or from both above and below.

The principle referred to above can be expanded by providing that the surface 30 is electrically contacted and embodied as an electrode for the plasma excitation by means of the oscillation generator 15. This would have the advantage of more-reliable self-sterilization, and if the sterilization course is long enough, even stopper regions that were shielded upon contact among stoppers 12 will have become sterile, and thus even bulk material can be treated with a relatively high throughput.

However, individualized treatment and conveying of the stoppers 12 is also conceivable, optionally with the installation of additional guide elements, but in that case those elements would also have to be subjected constantly to a plasma. If a plasma subjection from above and below the surface 30 is not contemplated, or cannot be adequately achieved, then in this case inversion of the stoppers 12, optionally multiple times, can be done by means of defined motions of the surface 30 itself, for instance by means of the eccentric motion device 31. The various positions of the stoppers 12 are shown in FIG. 5 by the two views of the surface 30, linked side by side as indicated by an arrow 32, which illustrate various stages in the conveying of the stoppers 12. However, the inversion of the stoppers 12 to be treated can also be done by additional devices, such as inverters, baffles, or the like.

Still other exemplary embodiments, not shown here, of conveyor devices for the stoppers 12 can be employed, which for instance also have a drum, and the drum can be provided with a spindle that is hollow on the inside; once again, suitably adapted conveyance from the inward-transfer region to the outward-transfer region can also be achieved.

An exemplary embodiment of FIG. 6 and FIG. 7 has a variant apparatus, with two rollers 40 and 41 rotating in the same direction for conveying the stoppers 12; the rollers 40 and 41 are offset somewhat in height from one another, and optionally, in a subsidiary variant not shown here, may also have a slight inclination. FIG. 6 shows a cross section and FIG. 7 a longitudinal section of this exemplary embodiment. The effect of the height offset of the rollers 40 and 41 is that a given stopper 12 is seated with its edge on one roller 41 while with its outside or inside it rests on the other roller 40. Because of the rotation of the rollers 40 and 41 in the same direction, the stopper 12 itself is set into rotation about its center axis and rolls at uniform speed along the rollers 40 and 41 from the inward transfer opening to the outward transfer opening, optionally reinforced by the inclination of the rollers 40 and 41.

By means of an inverting mechanism not specified in detail here, it is then possible next for the other side of the stopper 12 to be treated, on a second roller course, not shown but constructed in the same way. The plasma is generated in a comparable way as described above, by means of electrically conductive bodies 33 and 33a, which serve as antennas for broadcasting the electromagnetic field generated by the oscillation generator 15. Since along both of these course both the stopper 12 and the rollers 40 and 41 are moving constantly within the plasma 17, it is assured that a clear sterilization boundary exists between the inward- and outward-transfer regions.

In FIG. 8 and FIG. 9, a further exemplary embodiment can be seen, in which there is a quartz tube 43, which rotates about its axis and has a predetermined inclination a relative to the horizontal plane. Once again, FIG. 8 shows a cross section, while FIG. 9 shows a longitudinal section, through this exemplary embodiment. The stoppers 12 are fed individually into the opening 44, which here is located at the top, of the quartz tube 43, and they roll (arrow 49) toward the lower opening 46 during the rotation of the quartz tube 43 (arrow 45).

If here for example microwaves are projected in from outside the tube 43 for plasma generation, this can be in such a way that in the interior of the tube, a plasma is excited, which over time and over the course to be traversed in the tube acts on the stopper 12 on all sides. Locally generating the plasma in the interior of the tube 43 can be done by means of a suitable geometry of the apparatus and/or antenna, or by means of a pressure gradient or pressure jump between the inside and the outside. A plasma can also be locally generated in the interior of the tube 43 by providing that a more highly ignitable gas is fed into the interior than is present on the outside.

In the exemplary embodiment shown in FIGS. 8 and 9, a linear microwave source 48, such as a so-called gigatron, is disposed in the axis of the 43 and can likewise bring about a local generation of a plasma 47 in the required region of the quartz tube 43.

1. A method at least for sterilizing closing elements (12) for containers, in which
in at least one method step, in a low-pressure chamber or vacuum chamber (4), a plasma treatment is performed by excitation of an electromagnetic oscillation, and in which
the plasma (17; 47) is excited in the vicinity of the closing element (12) or of a group of closing elements (12) and of a conveyor device (10; 20; 30; 40, 41; 43) for the closing elements (12),
and the regions of the method step or method steps (12) to be sterilized, depyrogenized and/or annealed are treated between being transferred into and of our the chamber (4) by a motion in and through the plasma.

2. A method at least for sterilizing containers and their closing elements (12), in which
in at least one method step, in a low-pressure chamber or vacuum chamber (4), a plasma treatment is performed in common for the containers and the closing elements (12) by excitation of an electromagnetic oscillation, and in which
the plasma (17; 47) is excited in the vicinity of the closing element (12) or of a group of closing elements (12), of a conveyor device (10; 20; 30; 40, 41; 43) and of the containers, wherein
the regions of the closing element or closing elements (12) and containers to be sterilized, depyrogenized and/or annealed are treated between transfers into and out of the chamber (4) by a motion in and through the plasma.

3. A method at least for sterilizing containers and their closing elements (12), in which
in at least one method step, a plasma treatment is performed for the containers in one low-pressure chamber or vacuum chamber (4) and for the closing elements (12) in another low-pressure chamber or vacuum chamber (4), by excitation of an electromagnetic oscillation, and in which
the plasma (17; 47) is excited in the vicinity of one closing element or group of closing elements (12) and of a conveyor device (10; 20; 30; 40, 41; 43) and in the vicinity of the containers and a conveyor device, wherein
the regions of the closing element or closing elements (12) and containers to be sterilized, depyrogenized and/or annealed are treated between transfers into and out of the chambers (4, 5) by a motion in and through the plasma.

4. The method of claim 1, 2 or 3, characterized in that
before the containers and the closing elements (12) are transferred into the chambers (4), precleaning is done, and/or
after the containers and the closing elements (12) are transferred out of the chamber or chambers (4), filling of the containers and closure (7) of each of the containers with a respective closing element (12) are effected.

5. An apparatus for sterilizing closing elements (12) for containers by a method of one of the foregoing claims, characterized in that
in the chamber, there is a conveyor device (10; 30, 31; 40, 41, 42; 43), which effects a motion of the closing element or closing elements (12) for conveying them from the inward transfer opening (44) to the outward transfer opening (46) of the chamber and in the process through the plasma.

6. The apparatus of claim 5, characterized in that
the conveyor device (10) has a die (11) onto which a respective closing element (12) can be placed, and the die (11) acts as one electrical terminal for the plasma source (15), and a ring (14), on which an encompassing protrusion of the closing element (12) rests, acts as the other electrical terminal for the plasma source (15), and that
the die (11) is movable vertically such that in alternation, the closing element (12) rests either on the die (11) or on the ring (14).

7. The apparatus of claim 5, characterized in that
the conveyor device has one needle bearing or needle cushion (20) for each closing element (12), and that
the needles (21, 22), on which the closing element (12) rests, are movable essentially vertically in groups such that in alternation, the closing element (12) rests either on one group (21) or on the other group (22) of the needles.

8. The apparatus of claim 7, characterized in that
the needles on which the closing element rests are inclined at contrary angles in alternation in the groups (21, 22) and are some of them are movable essentially linearly and/or in terms of the angle of inclination such that an inclination and/or translational motion of the closing elements (12) can also be performed in the process.

9. The apparatus of one of claims 6-8, characterized in that
for retaining a plurality of closing elements (12), the ring (14) is a component of a perforated sheet, and the die (11) is a component of a die cushion, or the needles (20, 21, 22) are a component of an expanded needle cushion.

10. The apparatus of one of claims 7-9, characterized in that
the needles (20, 21, 22) or the dies (11) each act as an electrical terminal for the plasma source (15).

11. The apparatus of claim 5, characterized in that
the conveyor device is a plate conveyor (30) or a jigger table, with which in addition to the translational conveyance, a change in the position of the closing elements (12) relative to the plasma can be performed.

12. The apparatus of claim 11, characterized in that
in the case of a unilateral subjection of the closing elements (12) to the plasma, an inverting mechanism for the closing elements (12) is provided.

13. The apparatus of claim 5, characterized in that
the conveyor devices for the stoppers (12) is a drum with a spindle that is hollow on the inside or with guide baffles.

14. The apparatus of claim 5, characterized in that
the conveyor device is a roller conveyor, with closing elements (12) located between two rollers (40, 41), with which conveyor in addition to the translational conveyance in the direction of the roller axis, a change in the length of the closing elements (12) relative to the plasma (27) can be performed, and the rollers (40, 41) have an inclination to the horizontal plane.

15. The apparatus of claim 14, characterized in that
the rollers (40, 41), rotating in the same direction, are offset in height from one another by a predetermined amount.

16. The apparatus of claim 14 or 15, characterized in that
an inverting mechanism for the closing elements (12) is disposed at the end of a first roller assembly (40, 41), and on a second roller assembly, the other side of the closing elements (12) can be treated.

17. The apparatus of claim 5, characterized in that
the conveyor device is a quartz tube (43) that rotates about its longitudinal axis, whose longitudinal axis is inclined by a predetermined angular amount (a), so that the closing elements (12) can be conveyed during the rotation from the upper opening (44) to the lower opening (46) of the quartz tube (43) in such a way that a change in the location of the closing elements (12) relative to the plasma (47) which accomplishes an essentially rotational motion of the closing elements (12) during the conveyance from the inward transfer opening (44) to the outward transfer opening (46) in the quartz tube (43) can be performed.

18. The apparatus of claim 17, characterized in that
locally placeable generation of the plasma in the interior of the quartz tube (43) can be performed by means of a predetermined apparatus and/or antenna geometry, by means of a pressure gradient or pressure jump between the interior and the exterior of the quartz tube (43), or by means of feeding in a more highly ignitable gas in the interior of the quartz tube (43).

19. The apparatus of one of claims 5-18, characterized in that
the at least one plasma source is disposed in the interior of the chamber.

20. The apparatus of one of claims 5-19, characterized in that
the conveyor device is connected with an electrical terminal of the plasma source (15) or with a device for generating an electrical bias.

21. The apparatus of claim 19 in combination with claim 17 or 18, characterized in that
the at least one plasma source (48) is accommodated in the interior of the chamber along the longitudinal axis of the quartz tube (43).

22. The apparatus of one of claims 5-18, characterized in that
the plasma source is mounted on the outside of the chamber and is separated from the interior of the chamber by an arrangement by means of which the electromagnetic oscillations can be fed into the chamber and a pressure separation of the vacuum of the chamber from the pressure deviating from it outside the chamber can be accomplished.
